SEQUENCE LISTING (3 sequences)

SEQ ID NO: 1 - protein, 33 aa, Artificial Sequence
  Description of Artificial Sequence Synthetic polypeptide
LGTFWGDTLNCWMLSAFSRYARCLAEGHDGPTQ

SEQ ID NO: 2 - protein, 6 aa, Artificial Sequence
  Description of Artificial Sequence Synthetic peptide
FSRYAR

SEQ ID NO: 3 - protein, 7 aa, Artificial Sequence
  Description of Artificial Sequence Synthetic peptide
WMLSAFS

(19) United States
(12) Patent Application Publication
(10) Pub. No.: US 2020/0095280 A1
(43) Pub. Date: Mar. 26, 2020

(54) Methods of using GM6 in diagnosing and treating Alzheimers disease

(71) Applicant: Genervon Biopharmaceuticals LLC, PASADENA, CA (US)

(72) Inventors: Pui-Yuk Dorothy KO, MONTEREY PARK, CA (US); William R. SWINDELL, ATHENS, OH (US)

(73) Assignee: Genervon Biopharmaceuticals LLC, PASADENA, CA (US)

(21) Appl. No.: 16/473,066

(22) PCT Filed: Dec. 20, 2017
(86) PCT No.: PCT/US17/67479

§ 371 Date: Jun. 24, 2019

Related U.S. Application Data
(60) Provisional application No. 62/438,837, filed on Dec. 23, 2016.

(51) Int. Cl. C07K 7/06; G01N 33/68; A61K 9/00

(52) U.S. Cl. CPC C07K 7/06; G01N 33/6896; A61K 9/0019; A61K 38/00; G01N 2333/775; G01N 2800/2821; A61K 9/0053

(57) ABSTRACT

One aspect of this study was to provide a bioinformatic analysis to assess whether the MNTF-derived peptide known as GM6 alters the expression of genes associated with Alzheimer's disease. Gene expression analyses are performed using several gene expression profiling datasets generated by DNA microarray or RNA-seq technology. Our results show Alzheimer's disease-associated genes exhibit unique responses to GM6 treatment, impacting signaling pathways linked to core processes that underlie Alzheimer's disease onset and progression. The expression of one or more genes or gene variants of particular interest described herein. We show that ALS patients treated with GM6 exhibit significantly decreased abundance of plasma tau post-treatment (FIG. 1D). We also show that GM6 repressed MAPT mRNA in SH-5YSY cells (FIG. 2).

### RELATED APPLICATIONS

This application takes priority from U.S. Ser. No. 62/438,837, filed Dec. 23, 2016, entitled ‘Methods of using GM6 in treating Alzheimer's disease and in modulating Alzheimer's disease biomarkers’, by Pui-Yuk Dorothy Ko, incorporated by reference in its' entirety.

### FIELD

The field includes using the MNTF Factor known as GM6 for the diagnosing, monitoring, prognosing, preventing, delaying the onset, or treating Alzheimer's disease.

### BACKGROUND

The following includes information that may be useful in understanding the present inventions. It is not an admission that any of the information provided herein is prior art, or relevant, to the presently described or claimed inventions, or that any publication or document that is specifically or implicitly referenced is prior art.

Alzheimer's disease (AD) is a progressive dementia characterized by memory loss, brain atrophy, and neuronal death. The etiology is not fully understood, but appears to involve age-related accumulation of extracellular β-amyloid plaques and intracellular tau neurofibrillary tangles. To date, only five drugs have been approved by the U.S. Food and Drug Administration and available treatments have thus far offered AD patients limited benefit and efficacy (e.g., ACh inhibitors, NMDA receptor antagonists). Potentially, however, treatments under development for other neurodegenerative diseases will demonstrate efficacy for AD (i.e. “multipurpose” neurodegenerative drugs).

The isolation and characterization of two motoneuronotrophic factors (MNTF1 and MNTF2) from rat muscle tissues, as well as the subsequent cloning of a recombinant MNTF1-F6 gene derived from a human retinoblastoma cDNA library, is previously described in Applicant's prior U.S. Pat. No. 6,309,877. The MNTF1-F6 gene sequence encodes a 33 amino acid sequence referred to herein as SEQ ID NO:1 having the following amino acid sequence: LGTFWGDTLNCWMLSAFSRYARCLAEGHDGPTQ [SEQ ID NO:1].

The naturally occurring and recombinant MNTF1 polypeptides were shown to selectively enhance the survival in vitro of anterior horn motor neurons isolated from rat lumbar spinal cord explants. Photomicrographs of treated cultures exhibited neurite outgrowth of myelinated nerve fibers and a marked reduction in the growth of non-neuronal cells, e.g. glial cells and fibroblasts. Similarly, in vivo administration of MNTF1 to surgically axotomized rat peripheral nerves resulted in a markedly higher percentage of surviving motor neurons than untreated controls, which could be blocked by co-administration of anti-MNTF1 monoclonal antibody.

Further beneficial effects of MNTF1 were demonstrated in rats subjected to spinal cord hemi-section, repaired by a peripheral nerve autograft and implanted with MNTF1-containing gel sections in close proximity to the nerve graft junctions with spinal cord. MNTF1 treated animals exhibited greater numbers of surviving motor neurons, improved recovery of motor and sensory function, reduced inflammatory response (fewer infiltrating macrophages and lymphocytes) and reduced collagen-containing scar tissue formation at the site of the graft, normal Schwann cell morphology and normal myelinated and non-myelinated nerve fiber formation.

Two previously unrecognized overlapping domains within the MNTF1-F6 molecule that appear to be sufficient for the known biological activities of MNTF1 have now been identified. Each of these domains, designated herein as the “WMLSAFS” and “FSRYAR” domains, are sufficient to stimulate the proliferation of motor neuron derived cell lines in a manner similar to the MNTF1-F6 33-mer. Similarly, the “FSRYAR” domain is sufficient to direct selective reinnervation of muscle targets by motor neurons in vivo in a manner similar to the MNTF1-F6 33-mer. In addition, the “FSRYAR” domain provides an antigenic epitope sufficient to raise antibody that recognizes any MNTF peptide containing the “FSRYAR” sequence, including the MNTF1-F6 33-mer.

Novel peptides and composition from active fragments of MNTF that are capable of modulating viability and growth in neuronal cells, and to methods of modulating neuronal cell viability and growth employing the novel peptides and compositions, containing either a “WMLSAFS domain” or “FSRYAR domain”, which is sufficient for neurotrophic or neurotropic function is described in U.S. Pat. No. 7,183,373. The polypeptide domain demonstrated therein were sufficient for the selective maintenance and axonal regeneration of neuronal cells, and to peptides and/or molecules capable of mimicking their structure and/or function. Preferred embodiments of that invention comprise a peptide having the amino acid sequence: FSRYAR [SEQ ID NO:2], the sequence of GM6, also known as GM604 for ALS indication, as well as analogues thereof. Preferably such analogues are functional equivalents of the GM6 [SEQ ID NO:2].

GM6 encompasses the active domain of MNTF, an endogenous master neural growth regulator present during the fetal development phase when neurons are being created and reaching terminal synaptic targets. The fetal phase is the most intense and rapid period of human growth and development, especially within the CNS. In preclinical studies, treatment of GM604 in rodents demonstrated that it promotes neural regeneration and exhibits both trophic and tropic effects (Chau R M W et al. 1990 Neuronotrophic Factor. Chin J. Neuroanat. 6:129-138; Chau R M W et al. 1992. Muscle neurotrophic factors specific for anterior horn motoneurons of rat spinal cord. Recent Adv. Cell Mol Biol. 5:89-94; Yu J. et al. 2008. Motoneuronotrophic Factor analog GM604 reduces infarct volume and behavioral deficits following transient ischemia in the mouse. Brain Res. 1238:143-153; U.S. Pat. No. 7,183,373).

From the inventors in vitro and in vivo studies, we now understand that the MNTF 6 mer described herein and referred to as GM604 involves multiple mechanisms of action. GM6 binds to the insulin receptor and causes autophosphorylation of Tyr 1162/1163 of insulin receptor and IGF-1 (U.S. Pat. No. 8,986,676). GM6 also activates and modulates pathways through PI3K, as shown in the in vitro study with SH-SY5Y cells Parkinson Disease model, treatment with wortmannin (PI3K inhibitor) abrogated the effects of MNTF, implying effect through PI3K pathway (U.S. Pat. No. 8,673,852). The inventors have also shown the role of GM6 in anti-apoptosis, neurogenesis and anti-inflammation. In U.S. Pat. No. 8,673,852, the inventors showed that GM604 was able to penetrate the blood brain barrier.

This application reports the inventors results in the study of the role of GM6 in Alzheimer's disease, and the embodiments of the inventions provided herein are directed toward methods of diagnosing, monitoring, prognosing, and preventing, delaying the onset, or treating Alzheimer's Disease.

### BRIEF SUMMARY

The inventions described and claimed herein have many attributes and embodiments including, but not limited to, those set forth or described or referenced in this Summary. The inventions described and claimed herein are not limited to or by the features or embodiments identified in this Summary, which is included for purposes of illustration only and not restriction.

These and other aspects and embodiments of the inventions described and claimed herein will be apparent from and throughout the application and claims, all of which shall be considered to be a part of the written description thereof.

The inventions provided herein are directed toward methods of diagnosing, monitoring, prognosing, and preventing, delaying the onset, or treating Alzheimer's Disease.

Accordingly, one embodiment is directed to a method of preventing, delaying the onset, or treating Alzheimer's disease. The method includes the steps of: i) administering a MNTF peptide consisting of the amino acids FSRYAR [SEQ ID NO:2] (GM6) to a subject to inhibit or prevent Alzheimer's disease associated neuron loss, where the administration of GM6 inhibits mitochondria-mediated cell death by modulating one or more of the following genes: NDUFB1, NDUFA12, COX5A, ATPSO, COX7B, COX7A2, NDUFB7, NDUFB2, NDUFAB1, COX6C, NDUFC1, NDUFB6, NDUFB4, COX7C, UQCRH, NDUFA2, NDUFA8, NDUFS6, NDUFA7, NDUFB11, NDUFB10, NDUFS5, NDUFB9, NDUFA13, ATP5D, NDUFS8, NDUFA6, COX5B, NDUFS4, NDUFA1, COX6B1, NDUFS3, UQCRQ, PSENEN, NDUFA9, FADD, CALM3, COX8A, ATP5G3, PPP3CA, PLCB2, NDUFB3, COX4I1, CYC1, HSD17B10, CYCS, SDHB, CDK5, NDUFA5, APH1A, NDUFB5, COX7A2L, ATP5C1, ATP5F1, CACNA1F, MAPT, MAPK3, BAD, COX6B2, FAS, ATP5J, and UQCRB.

Another embodiment is directed to a method of preventing, delaying the onset, or treating Alzheimer's disease that comprises administering a MNTF peptide consisting of the amino acids FSRYAR (GM6) to a subject to inhibit or prevent Alzheimer's disease associated neuron loss, wherein administration of GM6 increases the catabolism of amyloid-precursor protein by modulating one or more of the following genes: CLU, SORL1, PICALM, GNAQ; IDE; ADAM10; PSEN1; PPP3CB; CASP3, CASP9, ITPR3, GSK3B, RTN3, BACE1, CDK5R1, ITPR2, EIF2AK3, ADAM17, CALML4, NCSTN, ATP5B, ATF6, APP, ATP2A1, PLCB4.

Another embodiment is directed to a method of preventing, delaying the onset, or treating Alzheimer's disease that comprises administering a MNTF peptide consisting of the amino acids FSRYAR (GM6) to a subject to inhibit or prevent Alzheimer's disease by limiting the expression and accumulation of Tau (MAPT).

In some embodiments of the preceding administration of GM6 inhibits mitochondria-mediated cell death by modulating a gene selected from wherein administration of GM6 inhibits mitochondria-mediated cell death by modulating a gene selected from: NDUFB1; NDUFA12; COXSA; ATPSO; COX7B; COX7A2; NDUFB7; NDUFB2; NDUFAB1; COX6C; NDUFC1; NDUFB6; NDUFB4; COX7C; UQCRH; NDUFA2; NDUFA8; NDUFS6; NDUFA7; NDUFB11; NDUFB10; NDUFS5; NDUFB9; NDUFA13; ATP5D; NDUFS8; NDUFA6; COX5B; NDUFS4; NDUFA1; COX6B1; NDUFS3; UQCRQ; PSENEN; NDUFA9; FADD; COX8A; ATP5G3; PLCB2; NDUFB3; COX4I1; NDUFB5. In some embodiments administration of GM6 inhibits mitochondria-mediated cell death by modulating a gene selected from NDUFB1; NDUFA12; COX5A; ATP50; and COX7B. In some embodiments administration of GM6 increases the catabolism of amyloid-precursor protein modulating a gene selected from GNAQ; IDE; ADAM10; PSEN1; PPP3CB; and CASP3. In some embodiments administration of GM6 increases the catabolism of amyloid-precursor protein modulating a gene selected from CLU, SORL1, PICALM.

Another embodiment is directed to a method of preventing, delaying the onset, or treating Alzheimer's disease that comprises administering a MNTF peptide consisting of the amino acids FSRYAR (GM6) to a subject to inhibit or prevent Alzheimer's disease associated neuron loss, wherein administration of GM6 increases dendrite morphogenesis, neurogenesis, or axon development by modulating one or more of the following genes: VLDLR, SORL1, C3orf17, STK11, RNF6, CNTN1, STK24, RELN, MAN2A1, TMEM106B, PICALM, CTNNA2, FARP1, APBB2, APP, PSEN1, ADAM10.

Another embodiment is directed to a method of preventing, delaying the onset, or treating Alzheimer's disease that comprises selecting a patient, ii) quantifying a biomarker for Alzheimer's disease in said patient, wherein the biomarker is selected from the group consisting of NDUFB1, NDUFA12, COX5A, ATP50, COX7B, COX7A2, NDUFB7, NDUFB2, NDUFAB1, COX6C, NDUFC1, NDUFB6, NDUFB4, COX7C, UQCRH, NDUFA2, NDUFA8, NDUFS6, NDUFA7, NDUFB11, NDUFB10, NDUFS5, NDUFB9, NDUFA13, ATP5D, NDUFS8, NDUFA6, COX5B, NDUFS4, NDUFA1, COX6B1, NDUFS3, UQCRQ, PSENEN, NDUFA9, FADD, CALM3, COX8A, ATP5G3, PPP3CA, PLCB2, NDUFB3, COX4I1, CYC1, HSD17B10, CYCS, SDHB, CDK5, NDUFA5, APH1A, NDUFB5, COX7A2L, ATP5C1, ATP5F1, CACNA1F, MAPT, MAPK3, BAD, COX6B2, FAS, ATP5J, and UQCRB; CLU, SORL1, GNAQ, IDE; ADAM10, PSEN1; PPP3CB; CASP3, CASP9, ITPR3, GSK3B, RTN3, BACE1, CDK5R1, ITPR2, EIF2AK3, ADAM17, CALML4, NCSTN, ATP5B, ATF6, ATP2A1, and PLCB4; VLDLR, C3orf17, STK11, RNF6, CNTN1, STK24, RELN, MAN2A1, TMEM106B, PICALM, CTNNA2, FARP1, APBB2, or APP iii) classifying the patient as in need of treatment for Alzheimer's disease if the quantity of said biomarker is determined to be sufficiently different from a predetermined level for a selected subject, iv) administering a MNTF peptide consisting of the amino acids FSRYAR [SEQ ID NO:2] (GM6) to a subject classified as in need of treatment to regulate one or more of the above biomarkers, wherein administration of GM6 inhibits mitochondria-mediated cell death, increases the catabolism of amyloid-precursor protein, or by limiting the expression and accumulation of Tau (MAPT), and v) correlating the regulation of the biomarker with an improvement in Alzheimer's disease progression.

Another embodiment is directed to a method of diagnosing Alzheimer's disease in a patient that comprises: detecting the level of expression of one or more genes or gene variants of apolipoprotein E (APOE) selected from the group consisting of: APOE e2, APOE e3, and APOE e4 in a biological sample from said patient, wherein differential expression of said one or more gene variants in the sample as compared to control levels of expression of said one or more genes or gene variants is indicative of Alzheimer's disease.

Another embodiment is directed to a method of treating or preventing Alzheimer's disease that comprises the steps of: i) administering a MNTF peptide consisting of the amino acids FSRYAR [SEQ ID NO:2] (GM6) to a subject to inhibit or prevent Alzheimer's disease associated neuron loss or dysfunction by modulating the expression of one or more genes or gene variants of apolipoprotein E (APOE) selected from the group consisting of: APOE e2, APOE e3, and APOE e4.

Another embodiment is directed to a method of diagnosing Alzheimer's disease in a patient that comprises: detecting the level of expression of one or more genes selected from the group consisting of: PLAU, NGFR, CACNA1G, CLU, and RYR3 in a biological sample from said patient, wherein differential expression of said one or more genes in the sample as compared to control levels of expression of said one or more genes is indicative of Alzheimer's disease.

Another embodiment is directed to a method of treating or preventing Alzheimer's disease that comprises the steps of: i) administering a MNTF peptide consisting of the amino acids FSRYAR [SEQ ID NO:2] (GM6) to a subject to inhibit or prevent Alzheimer's disease associated neuron loss or dysfunction by modulating the expression of one or more genes or genes selected from the group consisting of: PLAU, NGFR, CACNA1G, CLU, and RYR3.

Another embodiment is directed to a method of diagnosing Alzheimer's disease in a patient that comprises: detecting the level of expression of one or more genes selected from the group consisting of: DOCK2, VEGFA, IL6R, HMGB1 and PTK2B in a biological sample from said patient, wherein differential expression of said one or more genes in the sample as compared to control levels of expression of said one or more genes is indicative of Alzheimer's disease.

Another embodiment is directed to a method of treating or preventing Alzheimer's disease that comprises the steps of: i) administering a MNTF peptide consisting of the amino acids FSRYAR (GM6 to a subject to inhibit or prevent Alzheimer's disease associated neuron loss or dysfunction by modulating the expression of one or more genes or genes selected from the group consisting of: DOCK2, VEGFA, IL6R, HMGB1 and PTK2B.

Another embodiment is directed to a method of diagnosing Alzheimer's disease in a patient that comprises: detecting the level of expression of one or more genes selected from the group consisting of: COX412, NDUFS2, NDUFB8, NDUFS4 and COX10 in a biological sample from said patient, wherein differential expression of said one or more genes in the sample as compared to control levels of expression of said one or more genes is indicative of Alzheimer's disease.

Another embodiment is directed to a method of treating or preventing Alzheimer's disease that comprises the steps of: i) administering a MNTF peptide consisting of the amino acids FSRYAR (GM604) to a subject to inhibit or prevent Alzheimer's disease associated neuron loss or dysfunction by modulating the expression of one or more genes or genes selected from the group consisting of: COX412, NDUFS2, NDUFB8, NDUFS4 and COX10.

Another embodiment is directed to a method of diagnosing Alzheimer's disease in a patient that comprises: detecting the level of expression of one or more genes selected from the group consisting of: ABCA7, CLU, CR1, PICALM, PLD3, TREM2, and SORL1 in a biological sample from said patient, wherein differential expression of said one or more genes in the sample as compared to control levels of expression of said one or more genes is indicative of Alzheimer's disease.

Another embodiment is directed to a method of treating or preventing Alzheimer's disease that comprises the steps of: i) administering a MNTF peptide consisting of the amino acids FSRYAR (GM6) to a subject to inhibit or prevent Alzheimer's disease associated neuron loss or dysfunction by modulating the expression of one or more genes or gene variants selected from the group consisting of: ABCA7, CLU, CR1, PICALM, PLD3, TREM2, and SORL1.

### BRIEF DESCRIPTION OF THE DRAWINGS

FIG. 1 shows decreased plasma tau in ALS patients following 2 weeks GM6 treatment (Panels A-D, phase 2A study). (A) CSF tau. Patients were treated with GM6 for 2 weeks (visits 1-6) and followed for 10 weeks post-treatment (visits 7 and 8). (B) Plasma tau. (C) Baseline CSF tau (visit 1) versus CSF tau following the final GM6 treatment (visit 6). (D) Plasma tau following final GM6 treatment (visit 6) versus follow-up 31 days post-treatment (visit 7). In (C) and (D), patients with equal tau between visits lie along the diagonal line. P-values were obtained by comparing change in tau between visits in GM6- and placebo-treated patients (one-tailed t-test).

FIG. 2 shows 134 genes associated with the KEGG AD disease pathway (hsa05010) and their response to GM6 in SH-SY5Y cells. Genes shown grey font (*) were GM6-increased or GM6-decreased (FDR<0.10). Fold-change estimates (GM6/CTL) are indicated for each gene.

FIG. 3 illustrates AD-associated genes that are predominantly repressed by GM6 in SH-SY5Y cells and associated with mitochondria. (A, B) Simulation analyses. Sets of 134 SH-SY5Y-expressed genes were sampled at random. In (A), the histogram shows average FC among sets of 134 randomly sampled genes (arrow: observed average FC among 134 AD-associated genes). In (B), the histogram shows the average value of 2^{abs[log 2(FC)]} for each randomly sampled gene set, representing non-directional change in gene expression (arrow: observed value among 134 AD-associated genes). (C) GO BP terms enriched among AD-associated GM6-decreased genes (conditional hypergeometric test; left margin parentheses: number of GM6-decreased genes associated with each term; right margin: example GM6-decreased genes associated with each term).

FIG. 4 illustrates the KEGG AD disease pathway (hsa05010). Pathway components associated with GM6-increased or GM6-decreased genes are indicated (FDR<0.10; see legend).

FIG. 5 shows GO BP terms associated with GM6-increased genes linked to AD by genetic studies. (A) NHGRI-EBI GWAS catalog. (B) OMIM database. Left margin parentheses: number of GM6-increased genes associated with each term. Right margin: example GM6-increased genes associated with each term.

FIG. 6 shows GM6 is protective against toxic factors within ALS and AD patient CSF. Sprague Dawley rat cortical neuronal cells were treated with post-mortem CSF from control subjects or patients diagnosed with (A) ALS or (B) AD. Cell survival following 48 hrs. was assessed using MTT assays (n=5 per group). Groups that do not share the same letter differ significantly (P<0.05; Tukey HSD test).

FIG. 7 shows Genes associated with AD and their response to GM6 treatment (5+ database sources; directional test). AD-associated genes were identified and their average fold-change (GM6/CTL) was compared to randomly sampled gene sets. The arrow indicates the average fold-change (GM6/CTL) among AD-associated genes. The green histogram represents the distribution of average fold-change estimates in randomly sampled gene sets (10,000 random samples for each analysis). AD-associated genes used for this analysis include only those linked to AD based upon at least 5 database sources.

FIG. 8 shows Genes associated with AD and their response to GM6 treatment (4+ database sources; directional test). AD-associated genes were identified and their average fold-change (GM6/CTL) was compared to randomly sampled gene sets. The arrow indicates the average fold-change (GM6/CTL) among AD-associated genes. The green histogram represents the distribution of average fold-change estimates in randomly sampled gene sets (10,000 random samples for each analysis). AD-associated genes used for this analysis include only those linked to AD based upon at least 4 database sources.

FIG. 9 shows Genes associated with AD and their response to GM6 treatment (5+ database sources; non-directional test). AD-associated genes were identified and their average absolute fold-change [2\hat{ }abs(log 2(GM6/CTL))] was compared to randomly sampled gene sets. The arrow indicates the average absolute fold-change among AD-associated genes. The green histogram represents the distribution of average absolute fold-change estimates in randomly sampled gene sets (10,000 random samples for each analysis). AD-associated genes used for this analysis include only those linked to AD based upon at least 5 database sources.

FIG. 10 shows Genes associated with AD and their response to GM6 treatment (4+ database sources; non-directional test). AD-associated genes were identified and their average absolute fold-change [2\hat{ }abs(log 2(GM6/CTL))] was compared to randomly sampled gene sets. The arrow indicates the average absolute fold-change among AD-associated genes. The green histogram represents the distribution of average absolute fold-change estimates in randomly sampled gene sets (10,000 random samples for each analysis). AD-associated genes used for this analysis include only those linked to AD based upon at least 4 database sources.

FIG. 11 shows AD-associated genes most strongly altered by GM6 (Microarray). The figure shows AD-associated genes most strongly altered in SH-5YSY cells treated with GM6 for 48 hours. AD-associated genes with lowest p-values are shown. All genes were significantly altered by GM6 (FDR<0.10) with FC>1.50 or FC<0.67. Genes were linked to AD based upon 2 or more of 7 possible database sources.

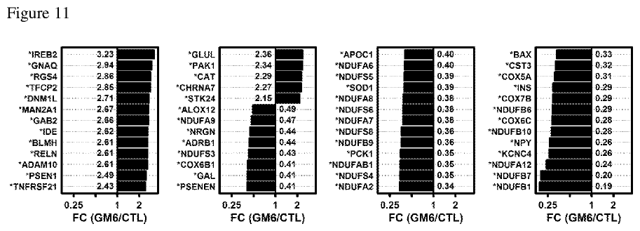

FIG. 12 shows AD-associated genes most strongly altered by GM6 (RNA-seq). The figure shows AD-associated genes most strongly altered in SH-5YSY cells (A) treated with GM6 for 6 hours (UM, RNA-seq), (B) treated with GM6 for 24 hours (UM, RNA-seq), (C) treated with GM6 for 48 hours (UM, RNA-seq), (D) treated with GM6 for 6 hours (SBH, 6 hours), and (E) treated with GM6 for 24 hours (SBH, 24 hours). AD-associated genes with lowest p-values are shown in each case. Genes with black bars were altered by GM6 with FC>1.50 or FC<0.67. An asterisk (*) is used to denote genes significantly altered with FDR<0.10 (left margin). Genes were linked to AD based upon 2 or more of 7 possible database sources.

FIG. 13 shows Gene ontology biological process (BP) terms associated with AD-associated genes altered by GM6 (UM, RNA-seq). (A) Gene ontology BP terms enriched with respect to GM6-increased AD-associated genes (FDR<0.10; pooled from the 3 UM RNA-seq experiments). Values in parentheses (left margin) indicate the number of GM6-increased genes in each category (right margin: exemplar genes most strongly induced by GM6). (B) Gene ontology BP terms enriched with respect to GM6-decreased AD-associated genes (FDR<0.10; pooled from the 3 UM RNA-seq experiments). Values in parentheses (left margin) indicate the number of GM6-decreased genes in each category (right margin: exemplar genes most strongly repressed by GM6).

FIG. 14 shows Gene ontology biological process (BP) terms associated with AD-associated genes altered by GM6 (SBH, RNA-seq). (A) Gene ontology BP terms enriched with respect to GM6-increased AD-associated genes (FDR<0.10; pooled from the 2 SBH RNA-seq experiments). Values in parentheses (left margin) indicate the number of GM6-increased genes in each category (right margin: exemplar genes most strongly induced by GM6). (B) Gene ontology BP terms enriched with respect to GM6-decreased AD-associated genes (FDR<0.10; pooled from the 2 SBH RNA-seq experiments). Values in parentheses (left margin) indicate the number of GM6-decreased genes in each category (right margin: exemplar genes most strongly repressed by GM6).

FIG. 15 shows KEGG pathways in AD diagram (hsa05010; SH-5YSY cells, Microarray, 48 hours GM6 treatment). Dark grey elements are associated with genes up-regulated in GM6-treated cells compared to CTL cells, and light grey elements are associated with genes down-regulated in GM6-treated cells compared to CTL cells. The color scale (bottom right) corresponds to log 2(GM6/CTL). An interactive version of this diagram is available online: http://www.genome.jp/kegg-bin/show_pathway?hsa05010

FIG. 16. shows KEGG pathways in AD diagram (hsa05010; SH-5YSY cells, UM RNA-seq, 6 hours GM6 treatment). Dark grey elements are associated with genes up-regulated in GM6-treated cells compared to CTL cells, and light grey elements are associated with genes down-regulated in GM6-treated cells compared to CTL cells. The color scale (bottom right) corresponds to log 2(GM6/CTL). An interactive version of this diagram is available online: http://www.genome.jp/kegg-bin/show_pathway?hsa05010

FIG. 17 shows KEGG pathways in AD diagram (hsa05010; SH-5YSY cells, UM RNA-seq, 24 hours GM6 treatment). Dark grey elements are associated with genes up-regulated in GM6-treated cells compared to CTL cells, and light grey elements are associated with genes down-regulated in GM6-treated cells compared to CTL cells. The color scale (bottom right) corresponds to log 2(GM6/CTL). An interactive version of this diagram is available online:

FIG. 18 shows KEGG pathways in AD diagram (hsa05010; SH-5YSY cells, UM RNA-seq, 48 hours GM6 treatment). Dark grey elements are associated with genes up-regulated in GM6-treated cells compared to CTL cells, and light grey elements are associated with genes down-regulated in GM6-treated cells compared to CTL cells. The color scale (bottom right) corresponds to log 2(GM6/CTL). An interactive version of this diagram is available online: http://www.genome.jp/kegg-bin/show_pathway?hsa05010

FIG. 19 KEGG pathways in AD diagram (hsa05010; SH-5YSY cells, SBH RNA-seq, 6 hours GM6 treatment). Dark grey elements are associated with genes up-regulated in GM6-treated cells compared to CTL cells, and light grey elements are associated with genes down-regulated in GM6-treated cells compared to CTL cells. The color scale (bottom right) corresponds to log 2(GM6/CTL). An interactive version of this diagram is available online: http://www.genome.jp/kegg-bin/show_pathway?hsa05010

FIG. 20 KEGG pathways in AD diagram (hsa05010; SH-5YSY cells, SBH RNA-seq, 24 hours GM6 treatment). Dark grey elements are associated with genes up-regulated in GM6-treated cells compared to CTL cells, and light grey elements are associated with genes down-regulated in GM6-treated cells compared to CTL cells. The color scale (bottom right) corresponds to log 2(GM6/CTL). An interactive version of this diagram is available online: http://www.genome.jp/kegg-bin/show_pathway?hsa05010

FIG. 21 Hypothesized mechanisms of action. This analysis identified AD-associated genes regulated by GM6 consistent with 4 potential mechanisms of action. The figure summarizes these mechanisms and lists GM6-regulated genes that may play a mediating role. All genes shown in the figure were regulated by GM6 in either or both RNA-seq experiments.

FIG. 22 shows that GM6 rapidly transits the Blood Brain Barrier. An ELISA assay was performed and results illustrated, with the supernatant from the brain homogenate detected GM6 at statistically significant levels at all doses (0.2 mg/kg and 2.0 mg/kg) compared to control (p=0.0001).

### DETAILED DESCRIPTION

### Definitions

A ‘biomarker’ is defined as any characteristic that is objectively measured and evaluated as an indicator of normal biological processes, pathogenic processes or pharmacogenomics processes to a therapeutic intervention. Both the FDA and EMA recognize the increasingly important role of biomarkers in the drug-development process. For devastating diseases, a search of disease-related biomarker can expedite the identification of a drug target. The use of disease-related biomarker as surrogate end points in clinical trials has expedited drug approval in oncology. The potential clinical benefits for disease-specific biomarkers include a more rapid and accurate disease diagnosis, and potential reduction in size and duration of clinical drug trials, which would speed up drug development. The application of biomarkers into drug development of Alzheimer's disease should both determine if a drug hits its proposed target (“target biomarkers”) and whether the drug alters the course of disease (“efficacy biomarkers”).

A ‘surrogate end point’ is defined as a biomarker that is intended to substitute for a known clinical end point, such as in the case of Alzheimer's disease. A surrogate endpoint is expected to predict benefit (or harm, or lack of benefit) based on epidemiologic, therapeutic, pathophysiologic or other scientific evidence. (Biomarkers Definitions Working Group (2001), Biomarkers and surrogate endpoints: preferred definitions and conceptual framework. Clin. Pharmacol. Ther.; 69(3):89-95) Such biomarkers are also frequently used to monitor disease progression in response to therapy.

### Pharmaceutical Compositions

The pharmaceutical formulations provided herein may further include, as optional ingredients, pharmaceutically acceptable carriers, diluents, solubilizing or emulsifying agents, and salts of the type that are available in the art. Examples of such substances include normal saline solutions such as physiologically buffered saline solutions and water. Specific non-limiting examples of the carriers and/or diluents that are useful in the pharmaceutical formulations of the present invention include water and physiologically acceptable buffered saline solutions such as phosphate buffered saline solutions pH 7.0-8.0. Suitable pharmaceutical carriers include, but are not limited to sterile water, salt solutions (such as Ringer's solution), alcohols, polyethylene glycols, gelatin, carbohydrates such as lactose, amylose or starch, magnesium stearate, talc, silicic acid, viscous paraffin, fatty acid esters, hydroxymethylcellulose, polyvinyl pyrolidone, etc. The pharmaceutical preparations can be sterilized and desired, mixed with auxiliary agents, e.g., lubricants, preservatives, stabilizers, wetting agents, emulsiers, salts for influencing osmotic pressure, buffers, coloring, and/or aromatic substances and the like which do not deleteriously react with the active compounds. They can also be combined where desired with other active substances, e.g., enzyme inhibitors, to reduce metabolic degradation.

Compounds provided herein may be formulated in a pharmaceutical composition, which may include pharmaceutically acceptable carriers, thickeners, diluents, buffers, preservatives, surface active agents, neutral or cationic lipids, lipid complexes, liposomes, penetration enhancers, carrier compounds and other pharmaceutically acceptable carriers or excipients and the like in addition to the compound.

Pharmaceutical compositions may also include one or more active ingredients such as interferons, antimicrobial agents, anti-inflammatory agents, anesthetics, and the like. Formulations for parenteral administration may include sterile aqueous solutions which may also contain buffers, liposomes, diluents and other suitable additives. Pharmaceutical compositions comprising the compounds provided herein may include penetration enhancers in order to enhance the alimentary delivery of the compounds. Penetration enhancers may be classified as belonging to one of five broad categories, i.e., fatty acids, bile salts, chelating agents, surfactants and non-surfactants (Lee et al., Critical Reviews in Therapeutic Drug Carrier Systems 8, 91-192 (1991); Muranishi, Critical Reviews in Therapeutic Drug Carrier Systems 7, 1-33 (1990)). One or more penetration enhancers from one or more of these broad categories may be included.

In certain embodiments, the pharmaceutical composition of the invention, can be administered locally, nasally, orally, gastrointestinally, intrabronchially, intravesically, intravaginally, into the uterus, sub-cutaneously, intramuscularly, periarticularly, intraarticularly, into the cerebrospinal fluid (ICSF), into the brain tissue (e.g. intracranial administration), into the spinal medulla, into wounds, intraperitoneally or intrapleurally, or systemically, e.g. intravenously, intraarterially, intraportally or into the organ directly, such as the heart.

The compounds provided herein may be administered parentally. It is sometimes preferred that certain compounds are combined with a pharmaceutically acceptable carrier or diluent to produce a pharmaceutical composition. Suitable carriers and diluents include isotonic saline solutions, for example phosphate-buffered saline. The composition may be formulated for parenteral, intramuscular, intracerebral, intravenous, subcutaneous, or transdermal administration. Uptake of nucleic acids by mammalian cells is enhanced by several known transfection techniques, for example, those that use transfection agents. The formulation which is administered may contain such agents. Example of these agents include cationic agents (for example calcium phosphate and DEAE-dextran) and lipofectants (for example Lipofectam™ and Transfectam™).

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated gloves, condoms, and the like may also be useful. Compositions for oral administration include powders or granules, suspensions or solutions in water or non-aqueous media, capsules, sachets or tablets. Thickeners, flavoring agents, diluents, emulsifiers, dispersing aids or binders may be desirable. Compositions for parenteral administration may include sterile aqueous solutions which may also contain buffers, diluents and other suitable additives. In some cases it may be more effective to treat a patient with an compound in conjunction with other traditional therapeutic modalities in order to increase the efficacy of a treatment regimen. As used herein, the term “treatment regimen” is meant to encompass therapeutic, palliative and prophylactic modalities.

Dosing can be dependent on a number of factors, including severity and responsiveness of the disease state to be treated, and with the course of treatment lasting from several days to several months, or until a cure is effected or a diminution of the disease state is achieved. Toxicity and therapeutic efficacy of compounds provided herein can be determined by standard pharmaceutical procedures in cell cultures or experimental animals. For example, for determining The LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissues in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. Dosing schedules can be calculated from measurements of drug accumulation in the body of the patient.

Suitable dosage amounts may, for example, vary from about 0.1 ug up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of compounds provided herein will be specific to particular cells, conditions, and locations. In general, dosage is from 0.01 mg/kg to 100 mg per kg of body weight, and may be given once or more daily, weekly, monthly or yearly, or even less frequently. In the treatment or prevention of certain conditions, an appropriate dosage level will generally be about 0.001 to 100 mg per kg patient body weight per day which can be administered in single or multiple doses. Compounds according to the invention (e.g. antibodies and binding fragments thereof) may be formulated into pharmaceutical compositions for administration according to well known methodologies. Pharmaceutical compositions may, for example, comprise one or more recombinant expression constructs, and/or expression products of such constructs, in combination with a pharmaceutically acceptable carrier, excipient or diluent. Such carriers will be nontoxic to recipients at the dosages and concentrations employed. A suitable dosage may be from about 0.01 μg/kg to about 1 g/kg body weight, typically by the intradermal, subcutaneous, intramuscular or intravenous route, or by other routes. A more typical dosage is about 1 μg/kg to about 500 mg/kg, with about 10 μg/kg, 100 μg/kg, 1 mg/kg, 10 mg/kg, 20 mg/kg, 50 mg/kg, 100 mg/kg, 200 mg/kg, 300 mg/kg, 400 mg/kg, and various ranges within these amount being still more typical for administration. It will be evident to those skilled in the art that the number and frequency of administration will be dependent upon the response of the host. “Pharmaceutically acceptable carriers” for therapeutic use are well known in the pharmaceutical art, and are described, for example, in Remingtons Pharmaceutical Sciences, Mack Publishing Co. (A.R. Gennaro edit. 1985). For example, sterile saline and phosphate-buffered saline at physiological pH may be used. Preservatives, stabilizers, dyes and even flavoring agents may be provided in the pharmaceutical composition. For example, sodium benzoate, sorbic acid and esters of p-hydroxybenzoic acid may be added as preservatives. Id. at 1449. In addition, antioxidants and suspending agents may be used. Id.

“Pharmaceutically acceptable salt” refers to salts of the compounds of the present invention derived from the combination of such compounds and an organic or inorganic acid (acid addition salts) or an organic or inorganic base (base addition salts). The compounds of the present invention may be used in either the free base or salt forms, with both forms being considered as being within the scope of the present invention.

However, pharmaceutical compositions provided herein may be in any form which allows for the composition to be administered to a patient. For example, the composition may be in the form of a solid, liquid or gas (aerosol). Typical routes of administration include, without limitation, oral, topical, parenteral (e.g., sublingually or buccally), sublingual, rectal, vaginal, and intranasal. The term parenteral as used herein includes subcutaneous injections, intravenous, intramuscular, intrasternal, intracavernous, intrathecal, intrameatal, intraurethral injection or infusion techniques. The pharmaceutical composition is formulated so as to allow the active ingredients contained therein to be bioavailable upon administration of the composition to a patient. Compositions that will be administered to a patient take the form of one or more dosage units, where for example, a tablet may be a single dosage unit, and a container of one or more compounds of the invention in aerosol form may hold a plurality of dosage units.

For oral administration, an excipient and/or binder may be present. Examples are sucrose, kaolin, glycerin, starch dextrins, sodium alginate, carboxymethylcellulose and ethyl cellulose. Coloring and/or flavoring agents may be present. A coating shell may be employed.

The composition may be in the form of a liquid, e.g., an elixir, syrup, solution, emulsion or suspension. The liquid may be for oral administration or for delivery by injection, as two examples. When intended for oral administration, preferred compositions contain, in addition to one or more binding domain-immunoglobulin fusion construct or expressed product, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

A liquid pharmaceutical composition as used herein, whether in the form of a solution, suspension or other like form, may include one or more of the following adjuvants: sterile diluents such as water for injection, saline solution, preferably physiological saline, Ringer's solution, isotonic sodium chloride, fixed oils such as synthetic mono or digylcerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

Compounds described herein can be used in diagnostics, therapeutics, prophylaxis, and as research reagents and in kits. Provision of means for detecting compounds of the invention can routinely be accomplished. Such provision may include enzyme conjugation, radiolabelling or any other suitable detection systems. Kits for detecting the presence or absence of compounds of the invention may also be prepared.

The compounds of the invention may also be used for research purposes. Thus, the specific activities or modalities exhibited by the compounds may be used for assays, purifications, cellular product preparations and in other methodologies which may be appreciated by persons of ordinary skill in the art.

Various aspects of the invention will now be described with reference to the following experimental section which will be understood to be provided by way of illustration only and not to constitute a limitation on the scope of the invention.

The following Examples are included for illustration and not limitation.

### Example 1

This study was performed to evaluate whether GM6 has effects consistent with an AD drug candidate. We show that short-term (2 week) treatment of amyotrophic lateral sclerosis patients with GM6 led to significant post-treatment declines in plasma tau (P=0.005). Consistent with this, treatment of SH-SY5Y cells with GM6 repressed expression of the MAPT mRNA encoding tau protein. Microarray analysis further indicated that AD-associated genes with mitochondrial functions are repressed by GM6 (e.g., NDUFB1, NDUFA12, COX5A). In contrast, several genes linked to AD by genetic studies and associated with amyloid precursor protein catabolism were up-regulated by GM6 (e.g., SORL1, PICALM). Finally, we show that GM6 partially rescues survival of rat cortical neurons treated with post-mortem CSF from AD patients. These findings suggest multiple mechanisms by which GM6 may be effective for AD, including the attenuation of mitochondrial dysfunction and reduction of neurofibrillary tangle or amyloid plaque accumulation. We report changes in tau protein from a recent phase 2A clinical trial with 12 ALS patients, microarray and bioinformatic analyses of the GM6 expression signature, and results from in vitro studies to assess protective effects of GM6 against cytotoxins within post-mortem CSF from AD patients.

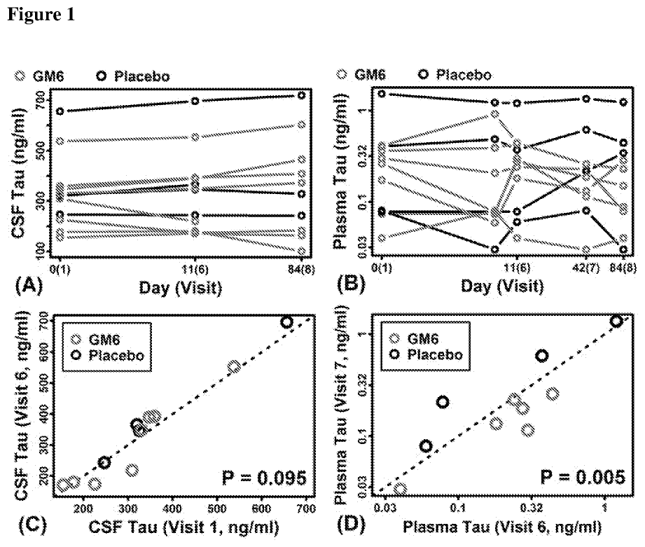

We examined tau protein in CSF and plasma from ALS patients in a phase 2A study, during which patients were given 6 doses of GM6 or placebo over 2 weeks (visits 1-6) and then monitored for 10 weeks post-treatment (visits 7 and 8) (FIG. 1). There was marginal evidence of decreased tau in CSF from GM6-treated patients following the final GM6 treatment (P=0.095; FIG. 1B). In plasma, all GM6-treated patients showed decreased tau during the post-treatment interval between visits 6 and 7 (FIGS. 1B and 1D; P=0.005). Short-term GM6 treatment in ALS patients thus led to a significant post-treatment decline in plasma tau (FIG. 1D).

Since lowering tau is one of the strategies to treat AD, the phase 2A plasma biomarker tau data shows GM6 has positive signal that it may be a therapeutic option to treat AD.

### Example 2—Bioinformatic Analysis and Mechanisms of Action

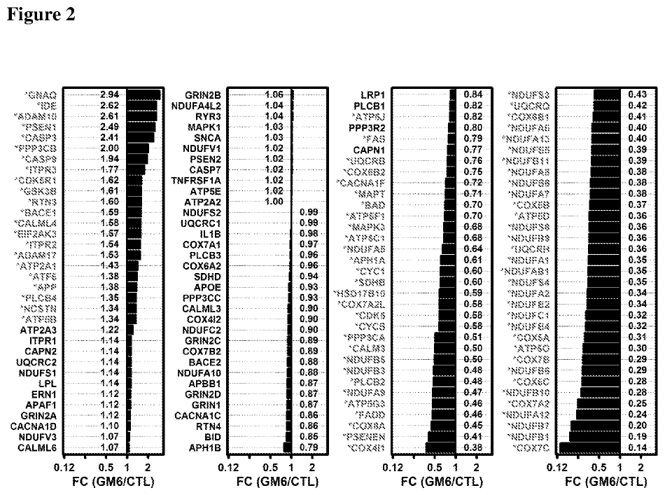

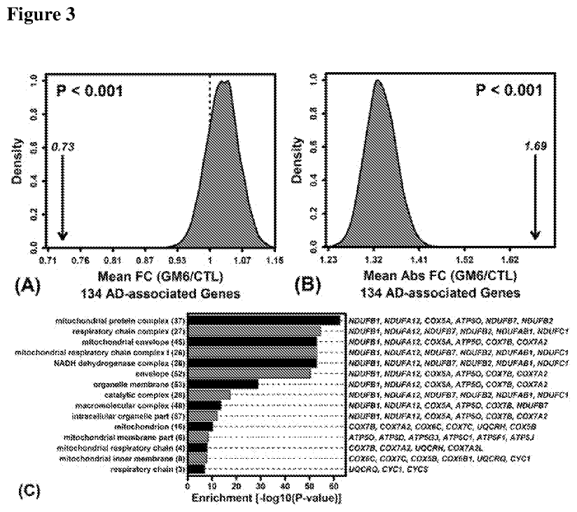

DNA microarrays were used to compare gene expression in GM6- and control-treated SH-SY5Y cells (n=2 replicates per treatment; 48 hrs. GM6 treatment). Based upon the KEGG AD disease pathway (hsa05010), we identified 171 AD-associated genes, of which 134 were expressed at detectable levels in SH-SY5Y cells (FIG. 2). Among these 134 AD-associated genes, 22 were GM6-increased but many more (62) were GM6-decreased (FIG. 2). As a group, simulation analyses showed that the 134 AD-associated genes were far more likely to be GM6-decreased, on average, as compared to randomly sampled sets of genes expressed by SH-SY5Y cells (FIG. 3A). The 62 AD-associated GM6-decreased genes were frequently linked to Gene Ontology (GO) Biological Process (BP) terms associated with mitochondria (FIG. 3C). Notably, the MAPT mRNA, which encodes the microtubule-associated protein tau, was down-regulated by GM6 (FC=0.71; FDR=0.023; FIG. 2).

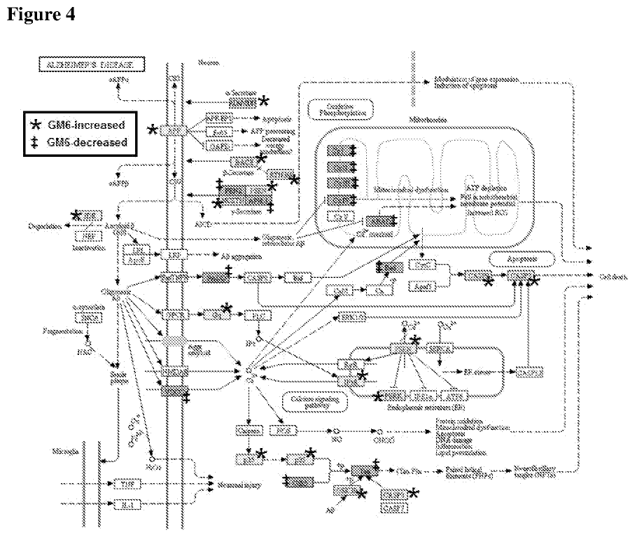

Inspection of the KEGG AD pathway diagram revealed that 5 of 6 mitochondrial components were down-regulated by GM6 (FIG. 4). Multiple upstream pathway components were also GM6-repressed, including genes encoding voltage-dependent calcium channels (CACNA1F) and Fas associated via death domain (FADD) (FIG. 4).

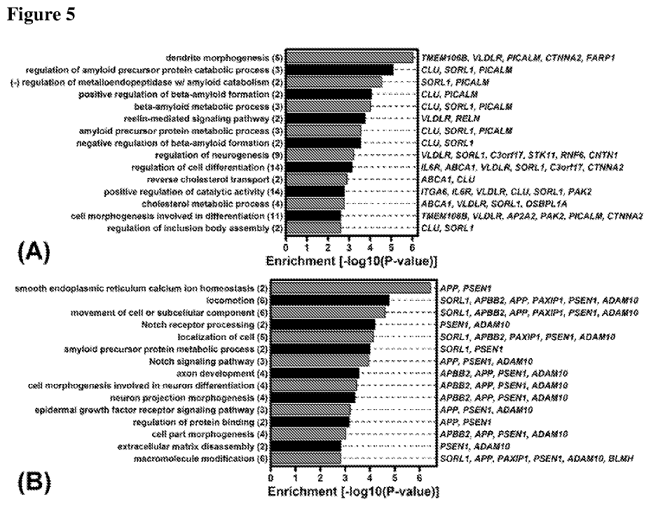

We used the NHGRI-EBI GWAS catalog and Online Mendelian Inheritance in Man (OMIM) database to identify genes linked to AD by genetic analyses. Such genes were disproportionately induced by GM6 (GWAS catalog: 202 genes, P=0.026; OMIM: 14 genes, P=0.002; n=2000 simulation trials). GM6-increased genes genetically associated with AD were involved in amyloid precursor protein catabolism and neuron morphogenesis (FIG. 5).

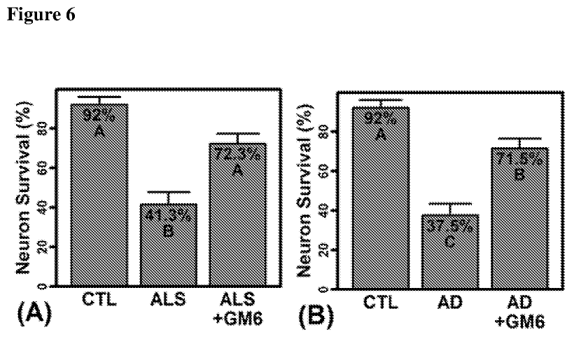

Finally, we evaluated whether GM6 could rescue rat cortical neurons following exposure to post-mortem CSF from ALS and AD patients (FIG. 6). Neuron cultures treated with CSF from control patients without neurodegenerative disease (48 hrs.) did not show impaired survival, but CSF from ALS or AD patients reduced survival by 60% (FIG. 6). For both diseases, GM6 provided a partial rescue, with significantly improved cell survival (FIG. 5). GM6 thus ameliorated cell death induced by cytotoxic factors in ALS and AD CSF, supporting the idea that GM6 may combat cell death due to multiple neurodegenerative diseases. These findings provide support and rationale for studies of GM6 as an AD drug candidate and suggest multiple mechanisms by which GM6 may slow neurodegeneration in this context.

In ALS patients treated with GM6, plasma tau decreased significantly post-treatment (FIG. 1D), and consistent with this, GM6 repressed MAPT mRNA in SH-5YSY cells (FIG. 2). Microarray analysis further indicated that mitochondrial genes associated with AD are predominantly down-regulated by GM6 (FIGS. 3 and 4), while genes linked to AD by genetic studies are up-regulated, including several associated with APP catabolism (e.g., SORL1; FIG. 5). We have additionally shown that GM6 partially rescues survival in rat cortical neurons cultured with post-mortem CSF from AD patients (FIG. 6).

These data suggest that GM6 may prevent AD-associated neuron loss by attenuating mitochondrial dysfunction (e.g., ROS generation, dysregulation of intrinsic apoptosis), limiting tau expression and accumulation (MAPT), and bolstering APP catabolism. Long-term data from randomized double-blinded placebo-controlled trials are needed to address the clinical efficacy of GM6 in AD patients.

### Additional Bioinformatic Analysis

Analyses were replicated with respect to 6 datasets generated from in vitro studies in which cultured SH-5YSY cells were treated with GM6 for varying lengths of time (Table 1).

TABLE 1
Expression profiling datasets. In all experiments, GM6 was applied
to cells at a concentration of 1 mg/ml in type I water.

| Dataset | Cell | | | GM6 Treatment | |
|---|---|---|---|---|---|
| No. | Type | Profiling | Company | Time | |
| 1^{a} | SH-SY5Y | Microarray | Sunny Bio | 48 | Hrs |
| 2^{b} | SH-SY5Y | RNA-seq^{c} | Sunny Bio | 6 | Hrs |
| 3^{b} | SH-SY5Y | RNA-seq^{c} | Sunny Bio | 24 | Hrs |
| 4^{b} | SH-SY5Y | RNA-seq^{c} | Sunny Bio | 48 | Hrs |
| 5^{b} | SH-SY5Y | RNA-seq^{d} | SBH Sciences | 6 | Hrs |
| 6^{b} | SH-SY5Y | RNA-seq^{d} | SBH Sciences | 24 | Hrs |

^{a}Experiments performed with n = 2 technical replicates per treatment.
^{b}Experiments performed with n = 3-5 biological replicates per treatment.
^{c}Sequencing performed by University of Michigan core sequencing facility.
^{d}Sequencing performed by Phalanx Biotech.

Dataset (1) was generated using DNA microarray technology and 2 technical replicates. For these data, differential expression analyses are performed based upon estimated fold-change (FC) values and FDR estimates. Since technical and not biological replicates were used in this experiment, FDR estimates are calculated for heuristic purposes but are not true FDR estimates (which would require biological replication).

Datasets (2)-(6) were generated using RNA-seq technology with experiments carried out by laboratories at Sunny Biodiscovery (Santa Paula, Calif.) or SBH Sciences (Natick, Mass.). For datasets (2)-(4), RNA-seq data were generated by core facilities at the University of Michigan (Ann Arbor, Mich.), while for datasets (5) and (6) RNA-seq was carried out by Phalanx Biotech (San Diego, Calif.).

Genes were linked to AD based upon an association from one or more of 7 database sources (Table 2). The databases used vary in terms of their criteria and stringency for identifying a gene as AD-associated. All analyses included in this report were performed using genes linked to AD based upon at least 2 of the 7 database sources. All genes highlighted in this report were thus linked to AD by at least 2 different sources, with many genes linked to AD based upon several sources (Table 2).

TABLE 2
Gene databases used to identify AD-associated genes. The
current bioinformatic analyses focus on those genes identified
with respect to at least 2+ database sources.

| | Pubmed | No. AD-associated |
|---|---|---|
| Source | Reference | Genes |
| 1. NHGRI-EBI GWAS Catalog^{a} | 27899670 | 459 |
| 2. MeSH Database^{b} | 25887539 | 4232 |
| 3. Disease Ontology^{c} | 25348409 | 429 |
| 4. DisGeNET^{d} | 27924018 | 176 |
| 5. KEGG Database^{e} | 27899662 | 171 |
| 6. eDGAR Database^{f} | 28812536 | 18 |
| 7. MalaCards^{g} | 27899610 | 186 |
| Common to 2+ sources | — | 786 |
| Common to 3+ sources | — | 239 |
| Common to 4+ sources | — | 73 |
| Common to 5+ sources | — | 29 |
| Common to 6+ sources | — | 8 |
| Common to 7 sources^{h} | — | 1 |

^{a}Database of genome-wide association studies (GWAS) reported since 2008 (www.ebi.ac.uk/gwas/).
^{b}Database of Medical Subject Headings (MeSH) terms based upon annotations of PubMed documents. AD-associated genes were identified based upon the MeSH term D000544 (https://www/ncbi.nlm.nih.gov/mesh).
^{c}Disease Ontology is a disease-centered database with genes organized according to disease etiology (http://www.disease-ontology.org). AD-associated genes were identified based upon the DO identifier 10652.
^{d}DisGeNET provides a comprehensive catalogue of genes and variants associated to human diseases (http://www.disgenet.org).
^{e}Kyoto Encyclopedia of Genes and Genomes (KEGG). AD-associated genes were identified based upon the KEGG pathway identifier hsa05010 (http://www.kegg.jp/).
^{f}Database of Disease-Gene Associations (eDGAR) (edgar.biocomp.unibo.it). The eDGAR database integrates gene-disease associations based upon the OMIM, HUMSAVAR and CLINVAR databases.
^{g}Integrated compendium of annotated diseases mined from 68 data sources (http://www.malacards.org/).
^{h}Apolipoprotein E (APOE) was common to all 7 database sources.

### (3) Results

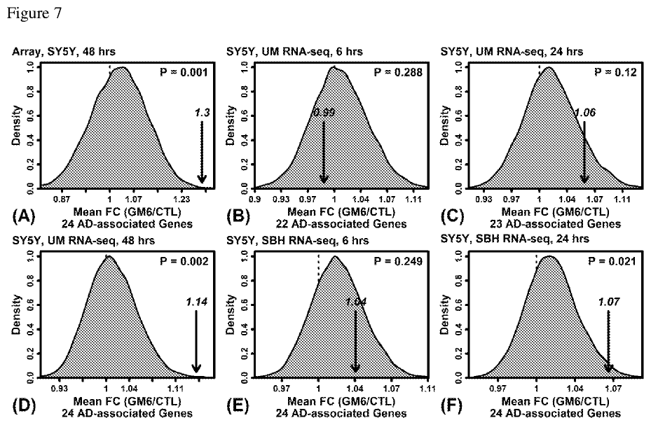

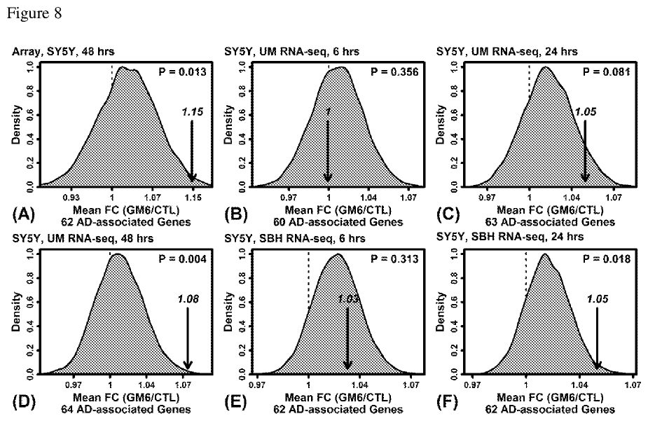

The average fold-change (GM6/CTL) of genes linked to AD based upon 5 or more sources was significantly increased with respect to three datasets (array 48 hr; UM RNA-seq 48 hr; SBH RNA-seq 24 hr; FIGS. 7 and 8). For example, with respect to the microarray study, 24 AD-associated genes linked to AD based upon 5+ sources were increased by 30%, which is a significantly stronger increase than observed in randomly sampled sets of 24 genes (P=0.001; FIG. 7B). Overall, a general trend was that treatment of SH-SY5Y cells with GM6 for 24 hours or longer tended to increase expression of AD-associated genes (Table 3).

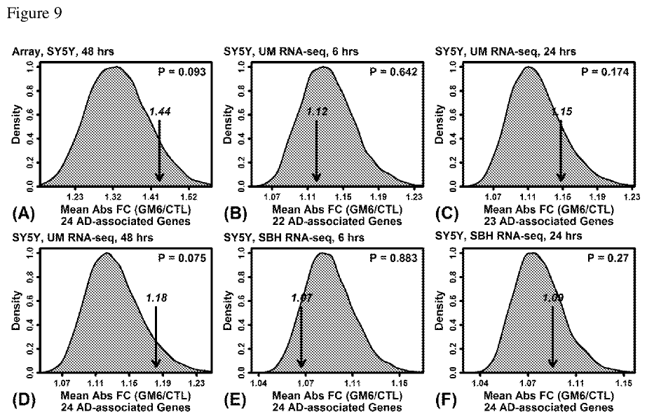

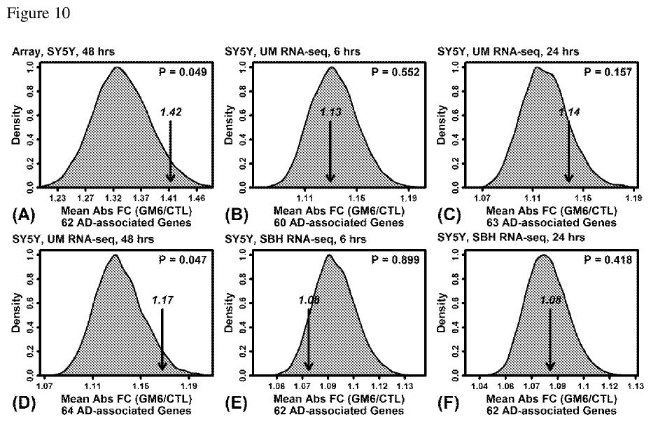

A second approach was to evaluate whether AD-associated genes were more responsive to GM6 stimulation compared to other genes, regardless of whether expression was elevated or repressed. The above analyses were thus repeated with average absolute fold-change [abs(log 2(GM6/CTL))] as the test statistic (FIGS. 9 and 10). This confirmed some trends identified above, but only with respect to genes linked to AD by 4+ database sources and for two datasets (array 48 hr; UM RNA-seq 48 hr; FIGS. 10A and 10D).

Gene set analyses described above evaluate the average effects of GM6 on AD-associated genes (i.e., gene set analyses; FIGS. 7-10). Table 3 summarizes p-values generated from these analyses and the two alternative analysis methods. Taken together (Table 3; FIGS. 7-10), these results suggest that GM6 has directional effects on the expression of AD-associated genes, tending to increase AD-associated genes rather than to decrease expression of AD-associated genes.

TABLE 3
Gene set analysis p-value summary. The table lists p-values
from simulation analyses evaluating whether average fold-change
estimates (GM6/CTL) of AD-associated genes differ significantly
from randomly sampled gene sets of the same size (FIGS. 7-10).

| | Directional Test^{a} | | Non-directional Test^{b} | |
|---|---|---|---|---|
| Comparison | 5+ sources | 4+ sources | 5+ sources | 4+ sources |
| 1. SH-SY5Y, array, 48 hr | P = 0.001* | P = 0.013* | P = 0.093 | P = 0.049* |
| 2. SH-SY5Y, UM RNA-seq 6 hr | P = 0.288 | P = 0.356 | P = 0.642 | P = 0.552 |
| 3. SH-SY5Y, UM RNA-seq 24 hr | P = 0.120 | P = 0.081 | P = 0.174 | P = 0.157 |
| 4. SH-SY5Y, UM RNA-seq 48 hr | P = 0.002* | P = 0.004* | P = 0.075 | P = 0.047* |
| 5. SH-SY5Y, SBH RNA-seq 6 hr | P = 0.249 | P = 0.313 | P = 0.883 | P = 0.899 |
| 6. SH-SY5Y, SBH RNA-seq 24 hr | P = 0.021* | P = 0.018* | P = 0.270 | P = 0.418 |

^{a}Directional test: Test evaluates whether AD-associated genes are more strongly increased or decreased in comparison to randomly sampled gene sets. The test statistic is average fold-change (GM6/CTL).
^{b}Non-directional test: Test evaluates whether AD-associated genes are more strongly altered (either direction) in comparison to randomly sampled gene sets. The test statistic is average absolute fold-change [abs(log2(GM6/CTL))].

It is instructive to examine trends among individual genes for which links to AD are most robustly supported by strong evidence. Overall, 4535 genes were identified as linked to AD based upon at least one database source, but only a small fraction of these were linked to AD based upon multiple sources (Data not shown). Only one gene, apolipoprotein E (APOE), was linked to AD based upon all 7 databases incorporated into the analysis (Table 2). There was a clear trend among RNA-seq findings demonstrating the GM6 increases APOE expression, with one experiment demonstrating a 2.86-fold increase following 48 hours of GM6 treatment (P=0.000697; data not shown).

Likewise there were 7 genes associated with AD based upon 6 or more database sources, and of these 6 were significantly altered by GM6 with respect to at least one comparison (P<0.05; PSEN2, PSEN1, APP, ADAM10, MAPT, SORL1; data not shown). Of these, the most frequently altered was microtubule associated protein tau (MAPT), which was significantly decreased with respect to the microarray dataset (FC=0.71; P=0.00721), but was in contrast significantly increased with respect to 3 comparisons associated with the RNA-seq datasets (23-38% increase with P≤0.000586; Data not shown).

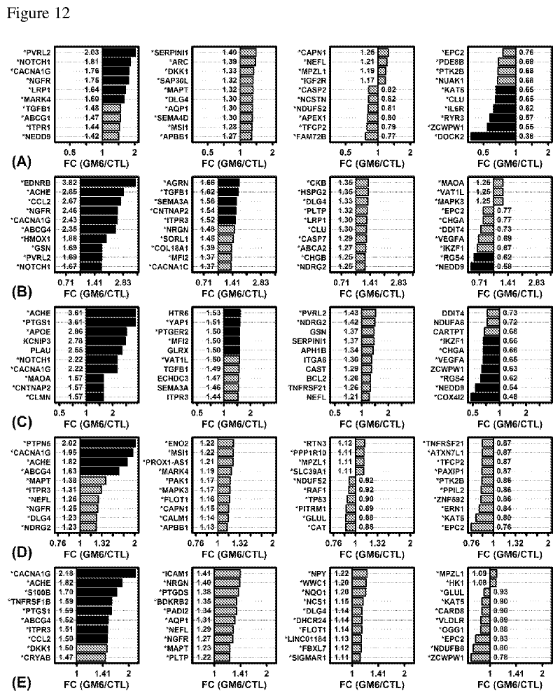

Among genes linked to AD based upon 2 or more database sources, the most strongly increased in the microarray dataset included IREB2, GNAQ and RGS4, and the most strongly decreased included NDUFB1, NDUFB7 and NDUFA12 (FDR<0.10; FIG. 5B). RNA-seq analyses demonstrated strongly increased expression of EDNRB (FC=3.82), ACHE (FC=3.61) and PTGS1 (FC=3.61), and strongly decreased expression of DOCK2 (FC=0.38), COX412 (FC=0.48) and NEDD9 (FC=0.54) (FIG. 12).

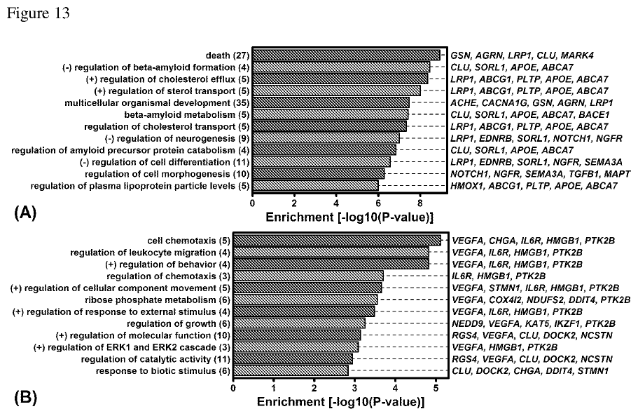

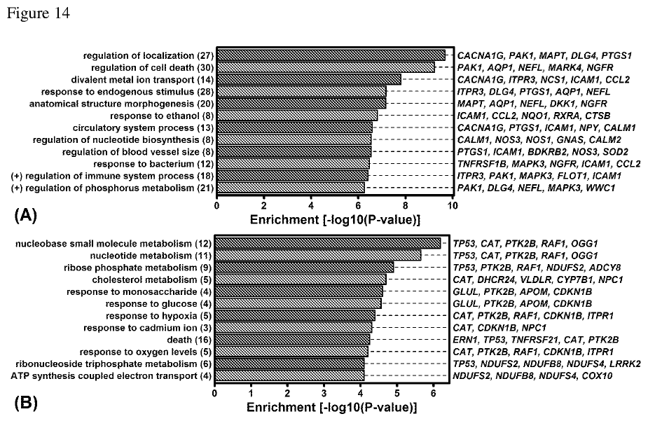

The UM and SBH RNA-seq datasets were the best replicated (n=3-5 per treatment) and thus further analyses were performed to characterize GM6-regulated genes identified from these experiments (FIGS. 13 and 14). With regard to the UM RNA-seq dataset, from among all 3 time points evaluated, a total of 64 GM6-increased and 30 GM6-decreased AD-associated genes were identified (FDR<0.10). The 64 GM6-increased genes were significantly associated with regulation of death, negative regulation of beta-amyloid formation, and positive regulation of cholesterol efflux (FIG. 12A), while the 30 GM6-decreased AD-associated genes were significantly associated with cell chemotaxis, regulation of leukocyte migration and positive regulation of behavior (FIG. 12B). Likewise, with respect to the SBH RNA-seq dataset, from the 2 time points evaluated, a total of 97 GM6-increased and 47 GM6-decreased AD-associated genes were identified (FDR<0.10). The 97 GM6-increased AD-associated genes were associated with regulation of localization, regulation of cell death and divalent metal ion transport (FIG. 14A), and the 47 GM6-decreased AD-associated genes were associated with nucleobase small molecule metabolism, nucleotide metabolism and ribose phosphate metabolism (FIG. 14B).

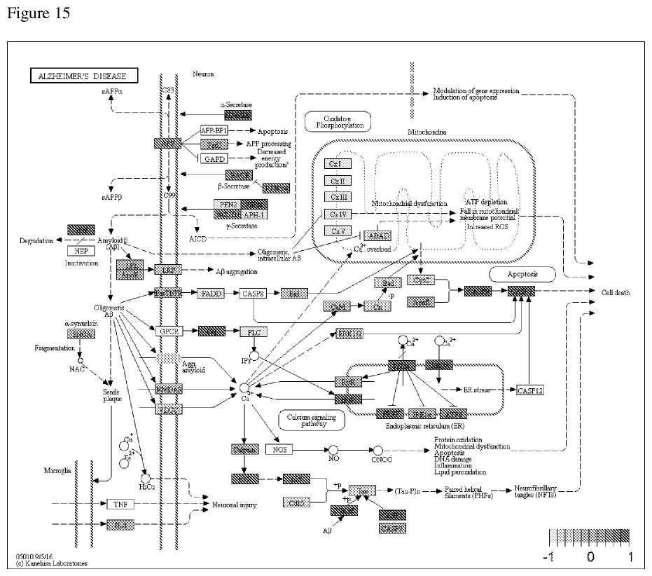

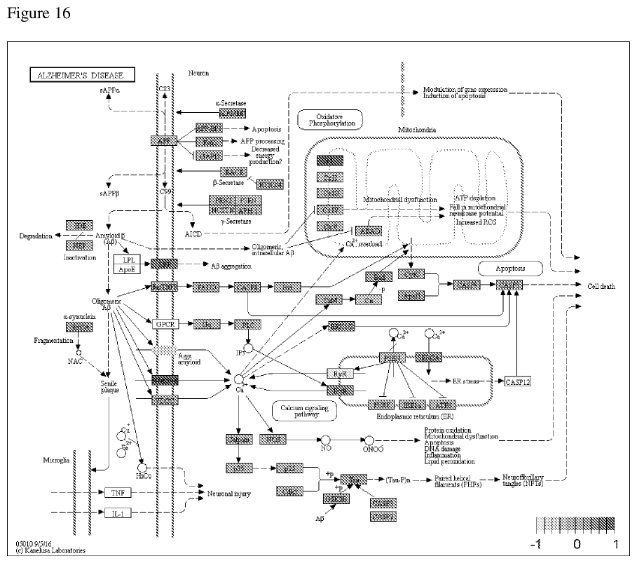

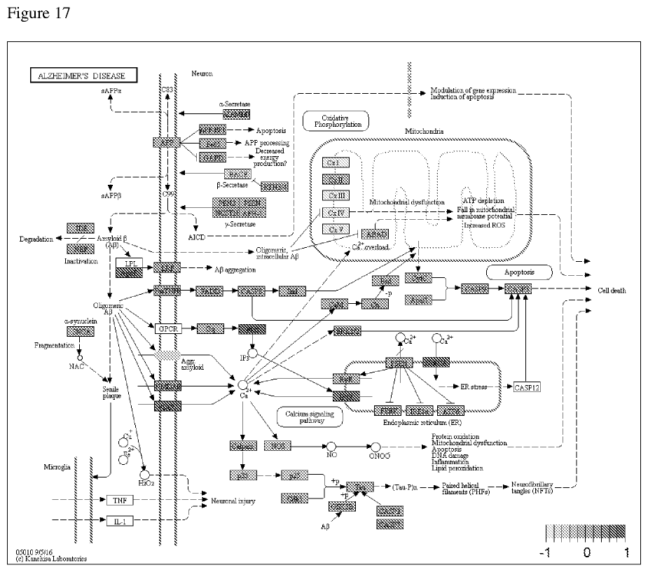

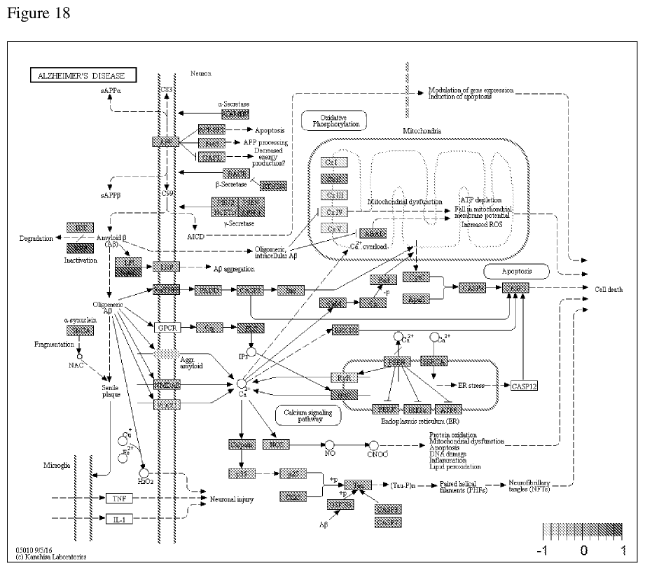

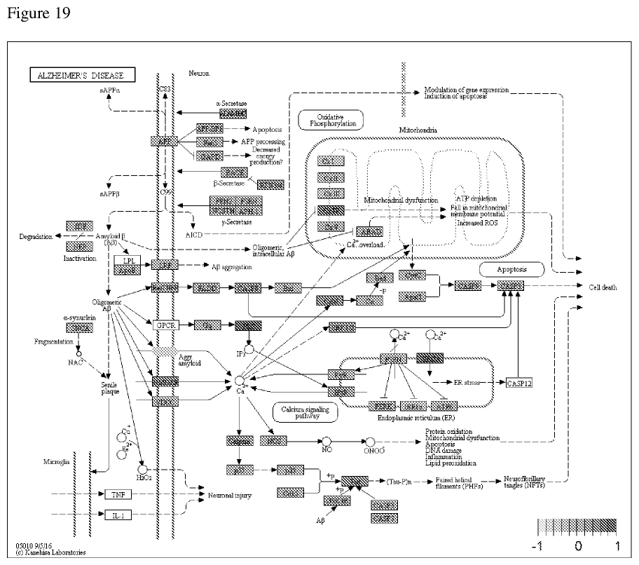

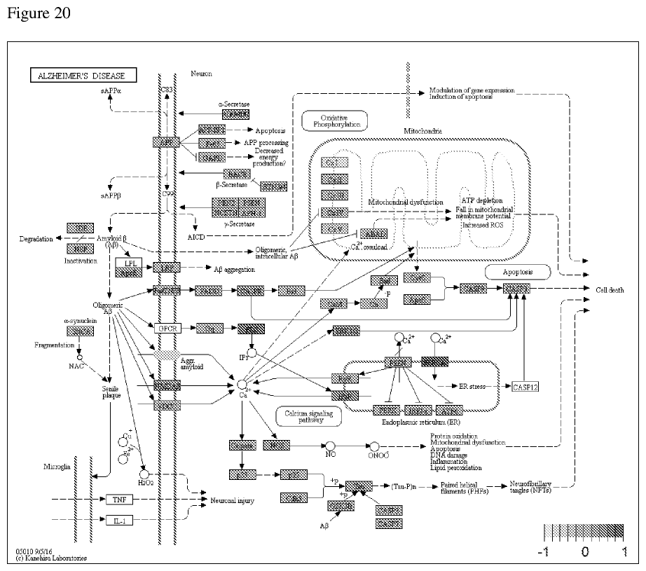

Patterns of GM6 gene regulation were visualized using the KEGG pathways in AD diagram (hsa05010). This diagram provides a gene mapping to pathways most commonly activated or inhibited in various forms of human AD, which collectively relate to induction of apoptosis, APP processing, modulation of gene expression, oxidative phosphorylation, neuronal injury, and neurofibrillary tangles (FIGS. 15-20). These diagrams revealed a number of upstream regulators significantly altered by GM6 (FIGS. 15-20). With regard to RNA-seq analyses, genes up-regulated by GM6 were associated with the upstream components LRP, NMDAR, ApoE, VDCC, NEP and PLC, and genes down-regulated by GM6 were associated with the upstream components BACE and RyR (FIGS. 15-20). There was a tendency for down-regulation of genes associated with mitochondrial components with respect to both microarray and RNA-seq datasets, with strongest effects observed for long-term treatment of SH-5YSY cells (i.e., ≥24 hours; FIGS. 15, 16, 17 and 20).

### (4) Conclusions

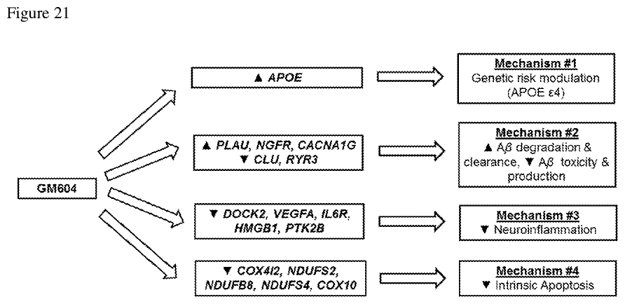

AD-associated genes regulated by GM6 were identified and functional properties of such genes were characterized. GM6 frequently increased expression of AD-associated genes in SH-5YSY, with stronger trends observed in cells treated 24 hours or longer (FIGS. 7-10). Modulation of AD-associated gene expression by GM6 was thus maximized by an extended treatment period (>24 hours). The analysis also identified GM6-regulated genes with potentially important effects on AD development and progression (FIG. 21). These genes were categorized with respect to 4 hypothesized mechanisms of action, including (1) genetic risk modulation, (2) inhibition of Aj3 production with augmentation of Aj3 degradation/clearance, (3) inhibition of neuroinflammation and (4) inhibition of intrinsic apoptosis cascades (FIG. 21).

The gene encoding apolipoprotein E (APOE) was significantly increased by GM6 in both RNA-seq studies and was elevated nearly 3-fold following 48 hours of GM6 treatment (FC=2.86; P=0.000697). APOE was the only gene linked to AD based upon all 7 database sources included in the current analysis, indicating a very high-confidence gene-disease association (Data not shown). Variants of the APOE gene are the most significant genetic risk factor for late-onset AD (PMID: 23296339). The gene encodes a chylomicron apoprotein mediating catabolism of lipoprotein constituents. The APOE e4 allele in particular has been identified as the pathological variant in AD, although it remains unclear whether the e4 variant augments AD risk through a gain of toxic function or a loss of protective function. Potentially, up-regulation of APOE expression by GM6 may have favorable compensatory effects in APOE e4 carriers. This suggests that effects of GM6 may be genotype-dependent, with different responses observed in those with the pathological e4 variant compared to those with the e2 or e3 variants. An important corollary of this possibility is that clinical studies evaluating effects of GM6 on AD should be designed and analyzed to allow for detection of genotype-specific treatment responses.

The role of extracellular Aβ plaques in AD pathology has been the subject of extensive debate (PMID: 28994715). In addition to their putative role as mediators of AD-related dementia, A13 plaques are believed to promote angiopathy that can increase long-term risk of intracranial bleeding (PMID: 29120920). The current analysis identified several mechanisms by which GM6 may attenuate Aβ plaque burden, including up-regulation of PLAU (plasminogen activator, urokinase), NGFR (nerve growth factor receptor), and CACNA1G (calcium voltage-gated channel subunit alphal G) as well as down-regulation of CLU (clusterin) and RYR3 (ryanodine receptor 3) (FIG. 15). PLAU encodes a secreted serine protease believed to contribute to Aβ plaques (PMID: 15615772), whereas NGFR has protective functions against Aβ toxicity (PMID: 25917367). CACNA1G encodes a voltage-sensitive calcium channel that contributes to calcium signaling involved in neurotransmitter release and AD appears to exacerbate age-related declines in CACNA1G expression (PMID: 24268883). Clusterin (CLU) has emerged as a therapeutic target in AD as well as other diseases and its abundance is elevated in the AD brain and believed to influence Aβ plaque abundance (PMID: 27978767). Finally, RYR3 encodes a ryanodine receptor that regulates intracellular calcium release, which may mediate Aβ plaque production and have acute pathological effects in later stages of AD (PMID: 22915123). The totality of these gene expression responses to GM6, therefore, may favor reduced accumulation of Aβ plaques during the course of cognitive aging.

Chronic neuroinflammation contributes to multiple neurodegenerative diseases of aging such as AD, Parkinson's disease and Multiple Sclerosis (PMID: 28790893). Regulators of neuroinflammation have therefore emerged as therapeutic targets for such diseases (PMID: 25652642). As a component of inflammatory reactions, DOCK2 (dedicator of cytokinesis 2) has been demonstrated to facilitate lymphocyte migration and is highly expressed in peripheral blood leukocytes as well as microglia. DOCK2 expression was down-regulated by 6 hours of GM6 treatment (FC=0.38; P=7.51e-06), although 24 hours of GM6 treatment increased DOCK2 expression (FC=2.09; P=0.0168). Effects of GM6 of DOCK2 expression were therefore time-dependent although the strongest response observed was down-regulated following the short-term 6 hour treatment time. This short-term response may be beneficial in the setting of AD, since DOCK2 regulates innate immune status of microglial cells within the AD brain (PMID: 19729484), and ablation of DOCK2 was shown to reduce Aβ plaque levels suggesting that DOCK2 may be a valid therapeutic target (PMID: 23318649).

Elevated rates of apoptosis appear to be characteristic of AD and may contribute to neuronal loss (PMID: 25322820). Such apoptotic neuronal death is mediated largely by cascades localized to mitochondria (PMID: 14555243), and mitochondria additionally appear to facilitate A1 plaque toxicity (PMID: 11387250). This study found significant down-regulation of COX412 (cytochrome c oxidase subunit 412) following 48 hours of GM6 treatment (FC=0.48; P=7.71e-07). This gene encodes a subunit for the enzyme cytochrome c oxidase (COX), which is the terminal enzyme of the mitochondrial respiratory chain. Other mitochondria-associated genes down-regulated by GM6 at varying time points include NDUFS2, NDUFB8, NDUFS4 and COX10 (FIG. 15). These results suggest that GM6 may have inhibitory effects on mitochondrial activity and/or abundance, which could contribute to decreased apoptosis through the intrinsic pathway and/or impaired production of reactive oxygen species (PMID: 19853657).

### A

### Example 3

GM6 can Transit Blood Brain Barrier as an Important CNS Therapeutic Treatment Strategy

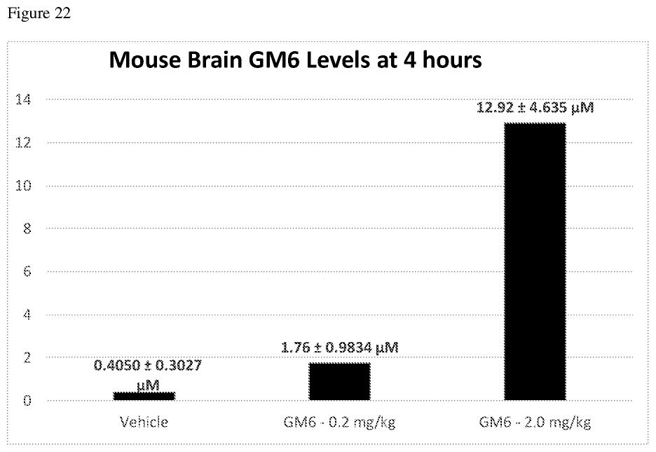

The ability to transit to the brain was tested. C57BL6 mice were injected with a single bolus IV tail vein injection of GM6 at 0.2 and 2.0 mg/kg. At four hours, the animals were sacrificed, and half of the brain was frozen for ELISA analysis. ELISA assay with the supernatant from the brain homogenate detected GM6 at statistically significant levels, at all doses in a dose dependent manner, compared to control (P=0.0001) (see FIG. 22).

### CONCLUSION

Our results show that ALS patients have plasma tau abundance that is decreased significantly by GM6 treatment (FIG. 1D). We also show that GM6 repressed MAPT mRNA in SH-5YSY cells (FIG. 2). Microarray analysis further indicated that mitochondrial genes associated with AD are predominantly down-regulated by GM6 (FIGS. 3 and 4), while genes linked to AD by genetic studies are up-regulated, including several associated with APP catabolism (e.g., SORL1; FIG. 5). We have additionally shown that GM6 partially rescues survival in rat cortical neurons cultured with post-mortem CSF from AD patients (FIG. 6). RNA-seq identified additional significantly up- or down-regulated AD related genes. These genes were categorized with respect to 4 hypothesized mechanisms of action, including (1) genetic risk modulation, (2) inhibition of Aβ production with augmentation of Aβ degradation/clearance, (3) inhibition of neuroinflammation and (4) inhibition of intrinsic apoptosis cascades. The ability of GM6 to reach the brain, and the neuroprotection of GM6 for neurons against toxic effects in the CSF of AD patients support the conclusion that GM6 is a potential therapeutic treatment for AD through the mechanisms of action outlined above.

1.-22. (canceled)

23. A method of increasing the catabolism of amyloid-precursor protein in a subject, the method comprising administering a MNTF peptide consisting of the amino acids FSRYAR (GM6; SEQ ID NO: 2) to the subject.

24. The method of claim 23, wherein the GM6 is administered in combination with another drug or therapy.

25. The method of claim 23, wherein the GM6 is administered intravenously.

26. The method of claim 23, wherein the GM6 is administered orally, subcutaneously, intranasally, or intramuscularly.

27. A method of up-regulating the expression of NGFR (nerve growth factor receptor), sortilin related receptor 1 (SORL1) and/or phosphatidylinositol binding clathrin assembly protein (PICALM) in a subject, the method comprising administering a MNTF peptide consisting of the amino acids FSRYAR (GM6; SEQ ID NO: 2) to the subject.

28. The method of claim 27, wherein the GM6 is administered in combination with another drug or therapy.

29. The method of claim 27, wherein the GM6 is administered intravenously.

30. The method of claim 27, wherein the GM6 is administered orally, subcutaneously, intranasally, or intramuscularly.

31. A method of reducing amyloid plaque accumulation, inhibiting neuroinflammation, and/or limiting the expression and accumulation of Tau (MAPT) in a subject, the method comprising administering a MNTF peptide consisting of the amino acids FSRYAR (GM6; SEQ ID NO: 2) to the subject.

32. The method of claim 31, wherein the GM6 is administered in combination with another drug or therapy.

33. The method of claim 31, wherein the GM6 is administered intravenously.

34. The method of claim 31, wherein the GM6 is administered orally, subcutaneously, intranasally, or intramuscularly.

35. A method of treating or preventing Alzheimer's disease in a patient, the method comprising the steps of:
i) quantifying a biomarker for Alzheimer's disease in said patient, wherein the biomarker is selected from the group consisting of NDUFB1, NDUFA12, COX5A, ATP5O, COX7B, COX7A2, NDUFB7, NDUFB2, NDUFAB1, COX6C, NDUFC1, NDUFB6, NDUFB4, COX7C, UQCRH, NDUFA2, NDUFA8, NDUFS6, NDUFA7, NDUFB11, NDUFB10, NDUFS5, NDUFB9, NDUFA13, ATP5D, NDUFS8, NDUFA6, COX5B, NDUFS4, NDUFA1, COX6B1, NDUFS3, UQCRQ, PSENEN, NDUFA9, FADD, CALM3, COX8A, ATP5G3, PPP3CA, PLCB2, NDUFB3, COX4I1, CYC1, HSD17B10, CYCS, SDHB, CDK5, NDUFA5, APH1A, NDUFB5, COX7A2L, ATP5C1, ATP5F1, CACNA1F, MAPT, MAPK3, BAD, COX6B2, FAS, ATP5J, and UQCRB; CLU, SORL1, GNAQ, IDE; ADAM10, PSEN1; PPP3CB; CASP3, CASP9, ITPR3, GSK3B, RTN3, BACE1, CDK5R1, ITPR2, EIF2AK3, ADAM17, CALML4, NCSTN, ATP5B, ATF6, ATP2A1, and PLCB4; VLDLR, C3orf17, STK11, RNF6, CNTN1, STK24, RELN, MAN2A1, TMEM106B, PICALM, CTNNA2, FARP1, APBB2, and APP;
ii) classifying the patient as in need of treatment for Alzheimer's disease if the quantity of said biomarker is determined to be sufficiently different from a predetermined level for a selected subject, and
iii) administering a MNTF peptide consisting of the amino acids FSRYAR [SEQ ID NO:2](GM6) to the patient classified as in need of treatment in step ii).

36. A method of diagnosing Alzheimer's disease in a patient, comprising: detecting the level of expression of one or more genes selected from the group consisting of: APOE e2, APOE e3, and APOE e4; PLAU, NGFR, CACNA1G, CLU, and RYR3; DOCK2, VEGFA, IL6R, HMGB1 and PTK2B; COX412, NDUFS2, NDUFB8, NDUFS4 and COX10; ABCA7, CLU, CR1, PICALM, PLD3, TREM2, and SORL1; in a biological sample from said patient, wherein differential expression of said one or more genes in the sample as compared to control levels of expression of said one or more genes is indicative of Alzheimer's disease.

37. The method of claim 36, further comprising treating the patient whose differential expression of said one or more genes is indicative of Alzheimer's disease, wherein the treating comprises administering GM6 to the patient.

38. The method of claim 36, wherein the GM6 is administered in combination with another drug or therapy.

39. The method of claim 36, wherein the GM6 is administered intravenously.

40. The method of claim 36, wherein the GM6 is administered orally, subcutaneously, intranasally, or intramuscularly.
